(19) United States
(12) Patent Application Publication
(10) Pub. No.: US 2008/0082075 A1
(43) Pub. Date: Apr. 3, 2008

(54) Absorbent article with a C-fold absorbent layer

(75) Inventor: Linda Morrell-Schwartz, Bensalem, PA (US)

Correspondence Address: RATNERPRESTIA, P O BOX 980, VALLEY FORGE, PA, 19482-0980, US

(73) Assignee: Tyco Healthcare Retail Services AG

(21) Appl. No.: 11/529,037

(22) Filed: Sep. 28, 2006

(51) Int. Cl. A61F 13/15

(52) U.S. Cl. 604/385.201; 604/385.01; 604/378

(57) ABSTRACT

Absorbent articles suitable for use as sanitary napkins, diapers, bladder control pads, or other absorbent products are provided. The absorbent articles include a cover layer, a barrier layer, an absorbent layer, and an acquisition layer. The absorbent layer and acquisition layer are interposed between the cover layer and the barrier layer. The absorbent layer has a substantially C-shaped configuration and the acquisition layer lines an inner surface of the absorbent layer and folded regions of the absorbent layer which are oriented towards the cover layer. Products according to the invention may provide any of several benefits, including high performance in terms of at least one of comfort, fit, cost, and producibility.

### FIELD OF THE INVENTION

The present invention relates to absorbent articles such as disposable feminine sanitary napkins, diapers, bladder control pads, and the like. More specifically, the invention relates to absorbent articles which provide a high liquid penetration rate and retention to allow the skin of a wearer to remain substantially dry after the article has become wet.

### BACKGROUND OF THE INVENTION

Absorbent articles such as disposable feminine sanitary napkins, diapers, bladder control pads, and the like, are known for their major function of absorbing and containing body exudates. Such articles are thus intended to prevent the soiling, wetting, or other contamination of clothing or other articles, such as bedding, that come in contact with the wearer. Absorbent articles typically have a basic structure that includes a liquid permeable topsheet, a liquid impermeable backsheet, and an absorbent layer positioned between the topsheet and the backsheet. Recently, super absorbent materials have been used in absorbent layers to improve absorbent capacity and provide a drier feel against a wearer's skin.

Current super absorbent materials retain large volumes of fluid, but generally have lower fluid absorption rates compared to other materials. As such, absorbent articles having super absorbent layers typically include acquisition layers which rapidly take up fluid and then release it into the absorbent layer. Acquisition layers further provide an insulating layer between the absorbent layer and the wearer to prevent contact between a wet absorbent layer and the wearer's skin. While many developments have been made in the art of absorbent articles having absorbent layers and acquisition layers, there remains a need for further improvements in terms of at least one of comfort, fit, cost, and producibility.

### SUMMARY OF THE INVENTION

In one aspect, the invention provides an absorbent article having a cover layer, a barrier layer, an absorbent layer, and an acquisition layer. The absorbent layer is interposed between the cover layer and barrier layer, and has a substantially C-shaped configuration defining a central-most region and folded regions. The acquisition layer is interposed between the cover layer and the barrier layer such that a central region of the acquisition layer is positioned between the central-most region of the absorbent layer and the cover layer, and other regions of the acquisition layer are positioned between the folded regions of the absorbent layer and the cover layer.

According to another aspect of the invention, an absorbent article having a cover layer, a barrier layer, an absorbent layer, and an acquisition layer is provided. The absorbent layer is interposed between the cover layer and barrier layer, and has a substantially C-shaped configuration defining a central-most region, folded regions, an inner surface and an outer surface. The acquisition layer is interposed between the cover layer and the barrier layer such that a central region of the acquisition layer is positioned adjacent the inner surface of the absorbent layer along the central-most region of the absorbent layer. Other regions of the acquisition layer extend adjacent the outer surface of the absorbent layer in the folded regions of the absorbent layer.

In another aspect of the invention, a method for producing an absorbent article is provided. The method includes the step of positioning an acquisition layer adjacent an absorbent layer such as by aligning an acquisition layer substantially parallel over an absorbent layer. The absorbent layer is then folded into a substantially C-shaped configuration such that the central region of the acquisition layer is positioned adjacent a central-most region of the absorbent layer and other regions of the acquisition layer are positioned adjacent folded regions of the absorbent layer. The absorbent layer and acquisition layer are then interposed between the cover layer and barrier layer such that the central region of the acquisition layer is positioned between the central-most region of the absorbent layer and the cover layer and other regions of the acquisition layer are positioned between the folded regions of the absorbent layer and the cover layer.

In yet another aspect of the invention, an additional method for producing an absorbent article is provided. The method includes the step of positioning an acquisition layer adjacent an absorbent layer. The absorbent layer is then folded into a substantially C-shaped configuration defining a central-most region, folded regions, an inner surface and an outer surface such that the central region of the acquisition layer is positioned adjacent the inner surface of the absorbent layer and extending other regions of acquisition layer adjacent the outer surface of the absorbent layer in the folded regions of the absorbent layer.

### BRIEF DESCRIPTION OF THE DRAWINGS

The features of the invention and the elements characteristic of the invention are set forth with particularity in the appended claims. The figures are for illustration purposes only and are not necessarily drawn to scale. The invention itself, however, may best be understood by reference to the detailed description which follows when taken in conjunction with the accompanying drawing in which:

FIG. 1 is a schematic view of an absorbent article according to an exemplary embodiment of the invention.

FIG. 2 is a schematic view of an absorbent article according to a second exemplary embodiment of the invention.

FIG. 3 is a top perspective view of an absorbent article according to another exemplary embodiment of the invention.

FIG. 4 is a cross sectional view of an absorbent article along the 4-4 line of the embodiment shown in FIG. 3.

FIG. 5 is a cross sectional view of an absorbent article according to yet another exemplary embodiment of the invention.

FIG. 6 is a cross sectional view of an absorbent article according to still another embodiment of the present invention.

### DETAILED DESCRIPTION OF THE INVENTION

The invention will next be illustrated with reference to the figures. Such figures are intended to be illustrative rather than limiting and are included herewith to facilitate the explanation of the present invention. The figures are not to scale, and are not intended to serve as engineering drawings.

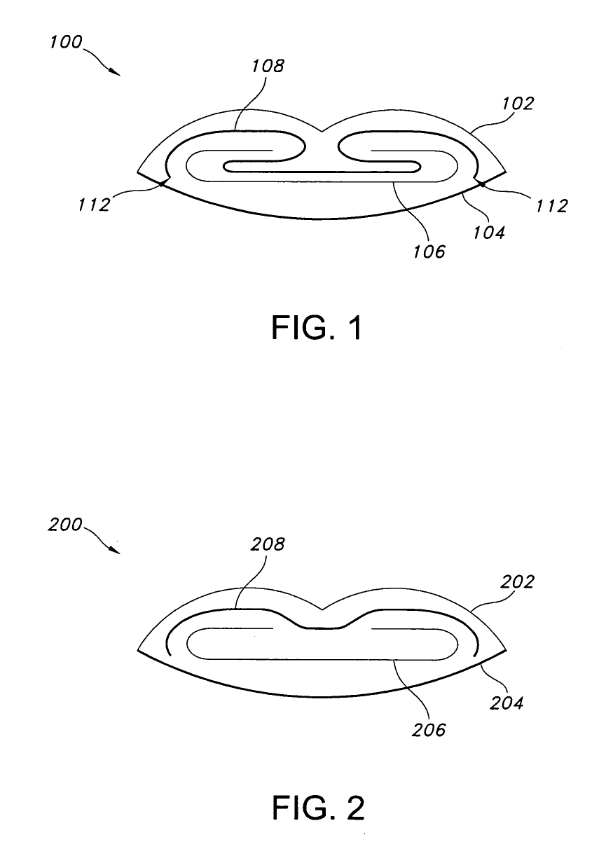

Referring generally to the drawings (FIGS. 1-6), the invention provides an absorbent article 100, 200, 300, 500, 600 having a cover layer 102, 202, 302, 502, 602, a barrier layer 104, 204, 304, 504, 604, an absorbent layer 106, 206, 306, 506, 606, and an acquisition layer 108, 208, 308, 508, 608. The absorbent core or layer 106, 206, 306, 506, 606 has a substantially C-shaped configuration and is interposed between the cover layer 102, 202, 302, 502, 602 and the barrier layer 104, 204, 304, 504, 604. The acquisition layer 108, 208, 308, 508, 608 is also interposed between the cover layer and barrier layer, and a central region of the acquisition layer 108, 208, 308, 508, 608 is positioned between the central-most region (generally designated by the numeral “1”) of the absorbent layer 106, 206, 306, 506, 606 and the cover layer 102, 202, 302, 502, 602. Other regions of the acquisition layer 108, 208, 308, 508, 608 are positioned in between folded regions 309, 509, 609 of the absorbent layer 106, 206, 306, 506, 606 and the cover layer 102, 202, 302, 502, 602. In one embodiment of the invention, the acquisition layer 108, 208, 308, 508, 608 is positioned adjacent or in contact with central-most region 1 of the absorbent layer 106, 206, 306, 506, 606, and the other regions of the acquisition layer are positioned adjacent or in contact with the folded regions 309, 509, 609 of the absorbent layer 106, 206, 306, 506, 606.

In further embodiments of the present invention, methods for producing an absorbent article 100, 200, 300, 500, 600 are provided. The methods include the step of positioning or aligning an acquisition layer adjacent or substantially parallel over an absorbent layer. The absorbent layer 106, 206, 306, 506, 606 is then folded or rolled into a substantially C-shaped configuration such that a central region of the acquisition layer 108, 208, 308, 508, 608 is positioned adjacent a central-most region 1 of the absorbent layer. Other regions of the acquisition layer are positioned adjacent to the folded regions of the absorbent layer. The absorbent layer and acquisition layer are then interposed between the cover layer 102, 202, 302, 502, 602 and the barrier layer 104, 204, 304, 504, 604.

Referring now to the drawing, in which like reference numbers refer to like elements throughout the various figures that comprise the drawing, FIG. 1 depicts a schematic view of an absorbent article 100 according to one embodiment of the present invention. The absorbent article includes a cover layer 102, a barrier layer 104, an absorbent core or layer 106, and an acquisition layer 108. When the absorbent article 100 is worn by a person, the cover layer 102 is arranged to face toward the body of the user (i.e. against the skin of the wearer) and the barrier layer 104 is arranged facing away from the wearer. The cover layer is superimposed over the barrier layer with the absorbent layer 106 and acquisition layer 108 interposed in between. The cover layer 102 is joined in any suitable manner, such as by adhesive bonding, heat sealing, ultrasonic bonding, or the like, to the barrier layer 104 around at least a portion of the periphery of the barrier layer 104.

The cover layer 102 is preferably liquid permeable in order to enable liquid to quickly and efficiently pass through the cover layer and into the acquisition layer 108. In particular, the cover layer may be selected from a variety of textile-like films and fabrics. Suitable fabrics include non-woven materials that are pervious to liquid, soft and pliable. Preferred non-woven materials include spun-bonded polypropylene, spunbonded polyethylene, and thermally bonded webs of staple fibers, preferably polypropylene shape or sheath/core bi-component fibers having a core of polyester or polypropylene and a sheath of polyethylene. To enhance the fluid control properties of the aforementioned liners, surfactants or wetting agents typified by Triton® X-100 and Triton® X-102 available from Rohm & Haas Company of Philadelphia, Pa. may be applied to the fluid receiving zones of the liner selectively, having the outer zones untreated to reduce migration of excreted fluid into the outer absorbent article regions, leading to leakage.

As further seen in FIG. 1, the barrier layer 104, which is arranged facing away from the wearer (i.e. away from the skin of the wearer), is preferably liquid impermeable and is optionally formed from a polypropylene film having a thickness of about 0.4 to 1.0 mils, as made available from Pliant Corporation, Williamsburg, Va. Other materials for forming the barrier layer may include polyethylene films, co-extruded films (polyethylene and ethylene vinyl acetate), co-polymer films (polyethylene/polypropylene), and polylaminates (polypropylene nonwoven and polyethylene film). Various multiple layer configurations or fiber denier variations may also be used. An example includes hydro-entangled non-woven webs, which may contain some cotton and/or rayon fibers blending in with thermal-plastic fibers. Cellulose fibers can also be blended in at small percentages to reduce cost. Still another example is a non-woven outer-cover made of stretchable or elastic materials, such as elastomeric composites of non-woven(s) and elastic membranes or a single layer of elastic material. The elastomeric composite can comprise an inner layer of pre-stretched extruded elastic film sandwiched between attached to a pair of non-woven webs. The non-woven webs may consist of spun-bond web, thermal-bond web, or a combination of the two. Preferably, the elastic film is made of synthetic rubber and the non-woven made of spun-bond polypropylene.

As seen with respect to the embodiment of the absorbent article 100 shown schematically in FIG. 1, the absorbent layer 106 which is interposed between the cover layer 102 and the barrier layer 104 has a substantially C-shaped configuration. The C-shaped configuration defines a central-most region and folded regions. An acquisition layer 108, which is also interposed between the cover layer 102 and the barrier layer 104, has a central region which is positioned between the central-most region of the absorbent layer 106 and the cover layer 102. Other regions of the acquisition layer 108, such as outer edge portions 112 of the acquisition layer 108, are positioned between the folded regions of the absorbent layer 106 and the cover layer 102.

The absorbent layer 106 is preferably made of materials including a super absorbent material, but may also be made of any suitable absorbent material, as well as combinations of different types of absorbent materials. For example, the absorbent layer 106 may be formed of a mixture of pulp fluff and super absorbent polymer (SAP) wrapped in a liquid permeable tissue wrap (not shown). Examples of SAP include polyacrylamides, polyvinyl alcohol, polyacrylates, various grafted starches, and the like. A desired super absorbent material is a cross-linked polysodium acrylate, which can be purchased from BASF Corporation of Portsmouth, Va., under the trademark ASAP® 2260. The super absorbent materials can be in various geometric forms, such as various shaped particles, fibers, foams, and layers.

The absorbent layer 106 may be of any shape, but is generally a flat rectangular sheet which is then rolled or folded into a C-shape configuration. The absorbent layer 106 is optionally a single layer or a layer formed by two or more sub-layers of absorbent material. In the C-shape configuration, the folded regions of the absorbent layer oriented towards the cover layer 102 form a natural container for holding larger volumes of liquid to be absorbed into the absorbent layer. The absorbent layer may be a single, integral absorbent structure, or can comprise a plurality of individual separate absorbent structures and/or absorbent materials that are operably assembled together. For instance, the absorbent layer may include two or more separate absorbent structures that are positioned side-by-side to the left and/or right of the central-most region or over the central-most region. The absorbent layer may also include two or more separate absorbent sublayers superposed with respect to one another. The absorbent layer may also include an air-laid non-woven web that contains super-absorbent particles and/or super-absorbent fibers, polymeric binder and cellulose pulp fibers.

In one exemplary embodiment, the absorbent core or layer is optionally sandwiched between two plies of tissue, is aligned on top (in the orientation shown in the figures) of the barrier layer 104 and adhered with construction adhesive. The tissue may typically have a basis weight of 17 gsm. Suitable tissues are available from Cellu Tissue Corporation, East Hartford, Conn. The absorbent layer 138 is typically centered along a central lengthwise direction or central axis of the absorbent article 100.

The central region of the acquisition layer 108 positioned between the central-most region of the absorbent layer 106 and cover layer 102, and other regions of the acquisition layer 108 positioned between the folded regions of the absorbent layer 106 and cover layer 102, function as an insulating layer between the absorbent layer 106 and cover layer 102. In other embodiments, it is contemplated that the central region of the acquisition layer 108 may be positioned either adjacent or in contact with the central-most region of the absorbent layer 106. Furthermore, other regions of the acquisition layer 108 may be positioned either adjacent or in contact with the folded regions of the absorbent layer 106.

As liquid penetrates the cover layer 102, the acquisition layer 108 is able to quickly absorb the liquid and transfer it to the absorbent layer 106. The acquisition layer 108 remains relatively dry even after liquid has penetrated the acquisition layer 108. The acquisition layer is generally composed of resilient fibrous webs that provide a small absorbent volume to rapidly take up liquids and then release it to the absorbent layer 106. This allows adequate time for super absorbent materials in the layer, when such super absorbent materials are optionally employed, to absorb the liquid. The thickness of the acquisition layer is preferably thin to provide the small absorbent volume and fast absorbency rate.

The acquisition layer 108 is typically composed of woven materials that are substantially nonabsorbent and hydrophobic. Suitable materials include fibrous webs and foams that may also include air-laid webs that contain polymeric binder fibers. Other suitable materials include polypropylene, polyethylene, and, preferably polypropylene sheath/core bi-component fibers having a core of polyester or polypropylene and a sheath of polyethylene. The acquisition layer 108 may be composed of any one of such materials, as well as combinations of different types of nonabsorbent materials.

As with the absorbent layer, the acquisition layer may include two or more separate acquisition structures that are positioned side-by-side or superposed with respect to one another. For instance, the acquisition layer may include two or more separate acquisition structures that are positioned side-by-side to the left and/or right of the central-most region or over the central-most region. The acquisition layer may also include two or more separate acquisition sublayers superposed with respect to one another.

The acquisition layer 108, existing as a mass of these materials, is able to accept liquids and allow passage of the liquid through its mass to be absorbed by the adjacent absorbent layer 106. Therefore, the mass of materials for the acquisition layer 108 may be absorbent, although the materials themselves are preferably not absorbent. Thus, the acquisition layer 108 which is preferably made of hydrophobic, nonabsorbent materials is able to accept large volumes of fluid into the interfiber spaces while the fibers themselves do not absorb any significant quantity of liquid. This allows the surface of the acquisition layer 108 to remain substantially or relatively dry even after liquid has penetrated the layer, and acts as an insulating layer between a relatively wet absorbent layer 106 and the wearer's skin.

As further seen in FIG. 1, when the absorbent article 100 is assembled, the acquisition layer 108 substantially follows the entire inner surface and portions of the outer surface of the absorbent layer 106 which is oriented towards the cover layer 102. It is contemplated that, in other embodiments, the acquisition layer 108 may line only a portion of the inner surface and portions of the outer surface of the absorbent layer 106 which is oriented towards the cover layer 102. In this configuration, a bottom outer surface of the absorbent layer 106 is exposed and adjacent to the barrier layer 104. This preferred feature provides full utilization of the acquisition layer 108 which absorbs liquid penetrating the cover layer 102 and then transfers it to the absorbent layer 106. Other benefits include less wasted acquisition layer material.

FIG. 2 depicts a schematic view of an alternative embodiment of an absorbent article generally designated by the numeral “200.”. As described above in FIG. 1, the absorbent article 200 has a cover layer 202, a barrier layer 204, an absorbent core or layer 206, and an acquisition layer 208. The acquisition layer 208 is configured to receive liquid through the cover layer 202 and then transfer it into the absorbent layer 206. The acquisition layer has a limited liquid-holding capacity compared to the absorbent layer 206 and remains substantially or relatively dry even after liquid has penetrated the acquisition layer. The central region of the acquisition layer 208 is positioned between the central-most region of the absorbent layer 206 and the cover layer 202, and other regions of the acquisition layer 208 are positioned between the folded regions of the absorbent layer 206 oriented towards the cover layer. In this configuration, the acquisition layer 208 is fully utilized and there is less wasted acquisition layer material.

The central region of the acquisition layer 208 is optionally positioned adjacent, yet spaced from the central-most region of the absorbent layer 206. Alternatively, the central region of the acquisition layer 208 is optionally positioned in direct contact the central-most region of the absorbent layer 206. Also, the other regions of the acquisition layer 208 are optionally positioned adjacent, or alternatively in direct contact with, the folded regions of the absorbent layer 206.

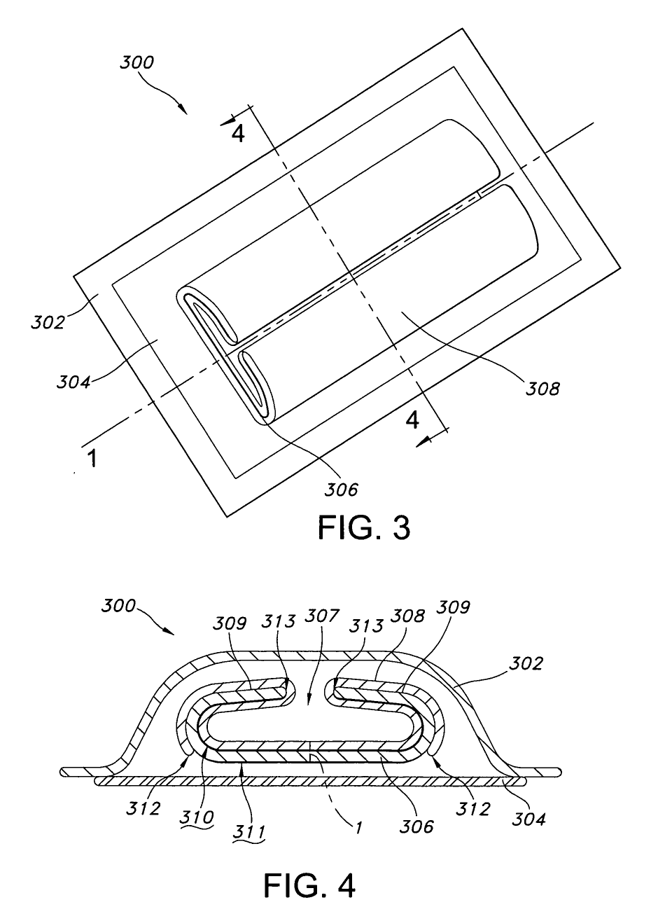

FIG. 3 depicts a top perspective view of the absorbent article 300 according to another exemplary embodiment of the present invention. The absorbent article 300 is designed to contact the wearer along the crotch area, and the article 300 forms a lowest point when worn by an individual standing upright. As described in FIG. 1, the absorbent article 300 includes a cover layer 302 superimposed over a barrier layer 304, with an absorbent layer 306 and acquisition layer 308 interposed in between the cover layer 302 and the barrier layer 304. When the absorbent article 300 is in place, the cover layer 302 is arranged to face toward the body of the user (i.e. against the skin of the wearer) and the barrier layer 304 is arranged facing away from the wearer.

The cover layer 302 is joined in any suitable manner, such as by adhesive bonding, heat sealing, ultrasonic bonding, or the like, to the barrier layer 304 around at least the periphery of the barrier layer 104 or a portion thereof. Although the cover layer 302 in FIG. 3 is shown with a surface area greater than barrier layer 304, it is contemplated that the cover layer 302 may have a surface area at least as large as the surface area of the barrier layer 304 or vice versa. As further shown in the depicted embodiment, the cover layer 302 and barrier layer 304 are substantially rectangular, but in an alternative embodiment the cover layer and/or barrier layer can be any suitable shape and dimension for a design of an absorbent article 300.

As seen with respect to the embodiment of the absorbent article 300 shown in FIG. 3, the absorbent layer 306 and acquisition layer 308 which are interposed between the cover layer and barrier layer are typically rectangular members that are centered in between the cover layer and the barrier layer. In other embodiments, the absorbent layer 306 and acquisition layer 308 may be of any shape or size. The absorbent layer 306 is preferably made of materials including a super absorbent material, but may also be made of any suitable absorbent material, as well as combinations of different types of absorbent materials as described previously in connection with FIG. 1.

FIG. 4 shows a cross sectional view of an absorbent article shown in FIG. 3 along the 4-4 line. The absorbent layer 306 and acquisition layer 308 are centered in between the cover layer 302 and the barrier layer 304. The absorbent layer 306 is shown with a substantially C-shaped configuration which defines a central-most region at 1, folded regions 309, an inner surface 310 and an outer surface 311. The acquisition layer 308 has a central region which is positioned adjacent to the inner surface 310 of the absorbent layer 306 along the central-most region 1 of the absorbent layer 306. In this embodiment, the acquisition layer 308 has a central region that actually contacts the inner surface 310 of the absorbent layer 306 along its central-most region 1.

The acquisition layer 308 also has regions, extending laterally from the central-most region, that actually contact the inner surface 310 of the absorbent layer 306 throughout the folded regions 309. Other regions of the acquisition layer 308, including additional regions extending laterally toward an outer edge 312 of the acquisition layer 308, extend adjacent to or optionally contact the outer surface 311 of the absorbent layer 306 in the folded regions 309 of the absorbent layer 306.

In its original pre-folded configuration (not shown), the absorbent layer 306 is flat and the layer 306 is then folded or rolled into a substantially C-shaped configuration. While the absorbent layer 306 is in its original flat configuration (not shown), an acquisition layer 308 having a surface area at least as large as the surface area of the absorbent layer 306 is optionally aligned substantially parallel over the layer 306. The absorbent layer 306 is then rolled or folded into a substantially C-shaped configuration, such that the absorbent layer has a central opening 307 and folded regions 309 oriented towards cover layer 302. Thus, the substantially C-shaped configuration of the absorbent layer 306 further defines a central opening 307 between the folded regions 309. More specifically, a central opening 307 is generally defined by a portion of the inner surface 310 of the absorbent layer 306 and the inner edges 313 of the folded regions 309 of the absorbent layer 306.

Excess or outwardly extending portions of the acquisition layer 308 near the absorbent layer 306 and along the lengthwise central axis 1 are then rolled or folded over the folded regions 309 of the absorbent layer 306 so as to cover the outer surfaces 311 of the absorbent layer 306 that are oriented towards the cover layer 302. In other words, other regions of the acquisition layer 308 are positioned between the folded regions 309 of the absorbent layer 306 and cover layer 302 in this manner. As a result, the acquisition layer 308 lines both a central region above the absorbent layer 306 and folded regions 309 of the absorbent layer 306 oriented towards the cover layer 302.

With respect to an absorbent layer 306 having an original flat pre-folded configuration (not shown), the absorbent layer 306 has a top surface that will become oriented towards the cover layer 302 and a bottom surface that will become oriented towards the barrier layer 304 when the absorbent layer 306 is interposed between the cover layer 302 and barrier layer 304. The top surface of the absorbent layer 306 will correspond to the inner surface 310, and the bottom surface will correspond to the outer surface 311.

When an acquisition layer 308 that has a surface area greater than the surface area of the absorbent layer 306 is aligned substantially parallel over the top surface of the absorbent layer 306, edge portions 312 of the acquisition layer 308 extend beyond the edges 313 of the absorbent layer 306, The absorbent layer 306 is then rolled or folded into a substantially C-shaped configuration, such that the top bottom surfaces of the flat layer become the inner surface 310 and outer surface 311 of a C-shaped layer 306, respectively. Therefore, when the absorbent layer 306 is rolled or folded into the substantially C-shaped configuration, the acquisition layer 308 covers, is optionally adjacent to, or optionally contacts the inner surface 310 of the absorbent layer 306 along at least the central-most region 1 of the absorbent layer 306. Additionally, other regions of the acquisition layer 308 are extended via the edge regions 312 of the acquisition layer 308 to contact or become adjacent to the outer surface 311 of the absorbent layer 306 in the folded regions 309 of the absorbent layer 306.

It is contemplated that, in other embodiments, the acquisition layer 308 may line only a portion of the inner surface and/or the outer surface of the folded regions 309 of the absorbent layer 306 which are oriented towards the cover layer 302. The folded regions 309 of the absorbent layer 306 which are lined with the acquisition layer 308 provide a holding area in which large amounts of liquid may pool from the central opening 307 and then be absorbed into the absorbent layer 306. A portion of the outer surface 311 of the absorbent layer 306, which is oriented towards and adjacent to the barrier layer 304, remains un-lined by the acquisition layer 308. When the absorbent article 300 is fully assembled, the portion of the outer surface 311 of the absorbent layer 306 which is oriented towards or adjacent to the barrier layer 304 optionally contacts the barrier layer 304 directly. In this configuration, the acquisition layer 308 is fully utilized and there is less wasted acquisition layer material.

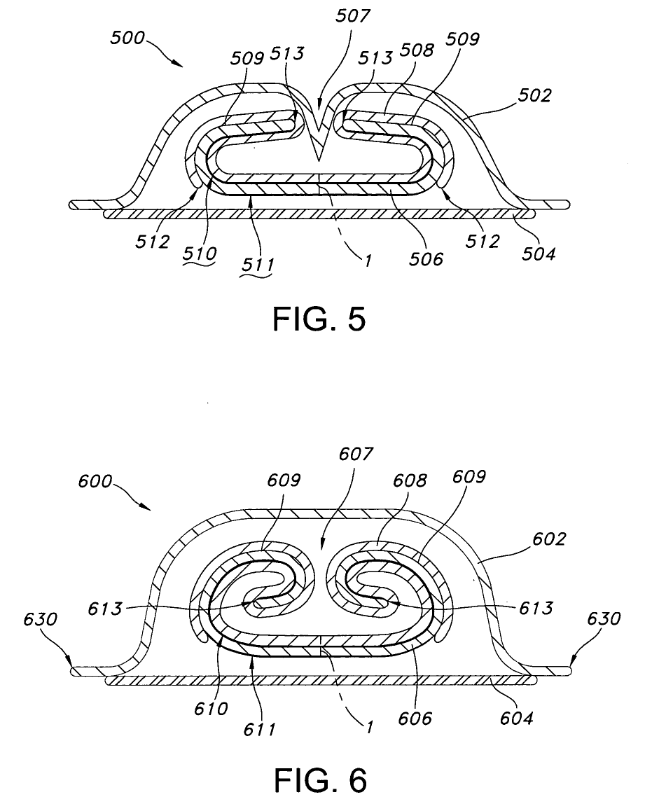

FIG. 5 shows a cross sectional view of an absorbent article according to yet another exemplary embodiment of the present invention. As described above in FIGS. 1, 3, and 4, the absorbent article 500 includes a cover layer 502, a barrier layer 504, an absorbent layer 506, and an acquisition layer 508. The absorbent layer 506 and acquisition layer 508 are interposed between the cover layer 502 and the barrier layer 504. The cover layer 502 is extends into a central opening 507 provided by the C-shaped absorbent layer 506. This provides a trough in which liquids would pool and be absorbed or transported by the acquisition layer 508 and absorbent layer 506 near the central opening 507.

With reference to FIG. 6, which depicts a cross sectional view of an absorbent article 600 in still another embodiment of the present invention, the absorbent article 600 includes a cover layer 602, a barrier layer 604, an absorbent layer 606, and an acquisition layer 608 as described above in FIGS. 1, 3, 4, and 5. The absorbent layer 606 and acquisition layer 608 are interposed in between the cover layer 602 and the barrier layer 604. The absorbent layer 606 is rolled or folded several times into a C-shaped configuration to provide multiple layers of the absorbent layer 606 sandwiched between the acquisition layer 608. In other word, the absorbent article 600 differs from absorbent article 500 in that an outermost edge portion 613 of the absorbent layer 606 extends downwardly in the folded region 609 and further extends outwardly toward an outer edge 630 of the absorbent article 600.

Liquid which penetrates the cover layer 602 and into the central opening 607 pools within the central region of the acquisition layer 608 to be absorbed by the absorbent layer 606. As further seen in FIG. 6, an inner surface 610 of the absorbent layer 606 is entirely lined by the acquisition layer 608, and a portion of the outer surface 611 of the absorbent layer 606 is adjacent to the liquid impermeable barrier layer 604. This provides full utilization of the acquisition layer 608.

It is expected that an absorbent article embodying features of the invention will exhibit improved performance in terms of absorbency rate. It is believed that the absorbency rate, measured in seconds, will be improved significantly because of the openness of the central-most region of the absorbent core, which provides a larger surface area or reservoir for the collection and absorption of fluid.

It is also expected that an absorbent article embodying features of the invention will exhibit improved utilization. It is believed that the capacity of fluid that the absorbent article can hold before leakage (measured using a mannequin test, for example) will approach the total capacity of the absorbent article (measured using an immersion test, for example). In other words, it is believed that a greater percentage of the absorbent article will be utilized before the article reaches its total capacity and before the article leaks. This means that a smaller article could be utilized while maintaining the same performance prior to leakage or that an article of a given size will exhibit improved performance prior to leakage.

In one actual example, total absorbent capacity testing and mannequin testing of an absorbent article with a C-fold absorbent layer as described above in connection with FIG. 1 revealed an actual total fluid holding capacity near the maximum theoretical capacity. This configuration provided improved absorbent efficiency and a near 100% utilization of the acquisition layer and absorbent layer. A summary of the results of the testing is provided in the following table:

| | |
|---|---|
| Total Capacity (gm) | 191.3 |
| (total capacity (gm) measured using an immersion test) | |
| Mannequin Test (s) | 27.1 |
| (time (s) until leakage of the absorbent article was detected using a | |
| mannequin test performed at 7 ml/s) | |
| Mannequin Total Capacity (gm) | 189.7 |
| (total capacity (gm) of the absorbent article before leakage was | |
| detected using the mannequin test) | |
| Utilization Rate (%) | 99.2 |
| (amount (%) of absorbent article utilized before Total Capacity is | |
| reached) | |

Accordingly, the Utilization Rate (the ratio of Mannequin Total Capacity to Immersion Total Capacity) is improved. The Utilization rate of exemplary embodiments of the invention is preferably greater than about 85%, more preferably greater than about 90%, and most preferably greater than about 95%.

Although the present invention has been particularly described in conjunction with specific embodiments, it is evident that many alternatives, modifications, and variations will be apparent to those skilled in the art. It is therefore contemplated that the appended claims will embrace any such alternatives, modifications, and variations as falling within the true scope and spirit of the present invention.

What is claimed:

1. An absorbent article comprising:
a cover layer;
a barrier layer;
an absorbent layer interposed between the cover layer and the barrier layer, wherein the absorbent layer has a substantially C-shaped configuration defining a central-most region and folded regions; and
an acquisition layer interposed between the cover layer and the barrier layer, wherein a central region of the acquisition layer is positioned between the central-most region of the absorbent layer and the cover layer, and other regions of the acquisition layer are positioned between respective ones of the folded regions of the absorbent layer and the cover layer.

2. The absorbent article according to claim 1, wherein the central region of the acquisition layer is positioned adjacent the central-most region of the absorbent layer.

3. The absorbent article according to claim 2, wherein the central region of the acquisition layer contacts the central-most region of the absorbent layer.

4. The absorbent article according to claim 1, wherein the other regions of the acquisition layer are positioned adjacent the folded regions of the absorbent layer.

5. The absorbent article according to claim 4, wherein the other regions of the acquisition layer contact the folded regions of the absorbent layer.

6. The absorbent article according to claim 1, wherein the cover layer has a surface area at least as large as a surface area of the barrier layer.

7. The absorbent article according to claim 1, wherein the acquisition layer has a surface area at least as large as a surface area of the absorbent layer.

8. The absorbent article according to claim 1, wherein the absorbent layer comprises a super absorbent material.

9. The absorbent article according to claim 1, wherein the cover layer comprises a liquid permeable material.

10. The absorbent article according to claim 1, wherein the barrier layer comprises a liquid impermeable material.

11. The absorbent article according to claim 1, wherein the acquisition layer is configured to transfer liquid into the absorbent layer.

12. The absorbent article according to claim 1, wherein the substantially C-shaped configuration of the absorbent layer further defines a central opening between said folded regions.

13. The absorbent article according to claim 12, wherein a portion of the cover layer extends into the central opening defined by the C-shaped configuration of the absorbent layer.

14. An absorbent article comprising:
a cover layer;
a barrier layer;
an absorbent layer interposed between the cover layer and the barrier layer, wherein the absorbent layer has a substantially C-shaped configuration defining a central-most region, folded regions, an inner surface and an outer surface; and
an acquisition layer interposed between the cover layer and the barrier layer, wherein a central region of the acquisition layer is positioned adjacent the inner surface of the absorbent layer along at least a portion of the central-most region of the absorbent layer, and other regions of said acquisition layer extend adjacent the outer surface of the absorbent layer in respective ones of the folded regions of the absorbent layer.

15. The absorbent article according to claim 14, wherein the central region of said acquisition layer contacts the inner surface of the absorbent layer along at least a portion of the central-most region of the absorbent layer.

16. The absorbent article according to claim 14, wherein the other regions of said acquisition layer contact the outer surface of the absorbent layer in respective ones of the folded regions of the absorbent layer.

17. The absorbent article according to claim 14, wherein regions of said acquisition layer extend adjacent the inner surface of the absorbent layer in respective ones of the folded regions of the absorbent layer.

18. The absorbent article according to claim 17, wherein regions of said acquisition layer contact the inner surface of the absorbent layer in respective ones of the folded regions of the absorbent layer.

19. The absorbent article according to claim 14, wherein the substantially C-shaped configuration of the absorbent layer further defines a central opening between said folded regions.

20. The absorbent article according to claim 14, wherein a portion of the cover layer extends into the central opening defined by the C-shaped configuration of the absorbent layer.

21. A method for producing an absorbent article comprising the steps of:
positioning an acquisition layer adjacent an absorbent layer;
folding the absorbent layer into a substantially C-shaped configuration such that a central region of the acquisition layer is positioned adjacent a central-most region of the absorbent layer and other regions of the acquisition layer are positioned adjacent respective folded regions of the absorbent layer; and
interposing the absorbent layer and the acquisition layer between a cover layer and a barrier layer such that a central region of the acquisition layer is positioned between the central-most region of the absorbent layer and the cover layer and the other regions of the acquisition layer are positioned between the respective folded regions of the absorbent layer and the cover layer.

22. The method for producing an absorbent article according to claim 21, wherein folding step comprises positioning the central region of the acquisition layer in contact with the central-most region of the absorbent layer.

23. The method for producing an absorbent article according to claim 21, wherein the folding step comprises positioning the other regions of the acquisition layer in contact with the respective folded regions of the absorbent layer.

24. The method for producing an absorbent article according to claim 21, wherein said folding step comprises providing a central region opening.

25. The method for producing an absorbent article according to claim 21, further comprising the step of coupling the cover layer to a periphery of the barrier layer.

26. A method for producing an absorbent article comprising the steps of:
positioning an acquisition layer adjacent an absorbent layer;
folding the absorbent layer into a substantially C-shaped configuration defining a central-most region, folded regions, an inner surface and an outer surface, thereby positioning a central region of the acquisition layer adjacent the inner surface of the absorbent layer along at least a portion of the central-most region of the absorbent layer and extending other regions of the acquisition layer adjacent the outer surface of the absorbent layer in respective ones of the folded regions of the absorbent layer.

27. The method for producing an absorbent article according to claim 26, further comprising the step of:
interposing the absorbent layer and the acquisition layer between a cover layer and a barrier layer, thereby positioning the central region of the acquisition layer between the central-most region of the absorbent layer and the cover layer and positioning the other regions of the acquisition layer between the respective folded regions of the absorbent layer and the cover layer.

28. The method for producing an absorbent article according to claim 26, wherein said positioning step comprises extending edge regions of the acquisition layer beyond edges of the absorbent layer.

29. The method for producing an absorbent article according to claim 28, further comprising the step of:
extending the edge regions of the acquisition layer adjacent the outer surface of the absorbent layer in respective ones of the folded regions of the absorbent layer.

30. The absorbent article according to claim 1 having a Utilization Rate greater than about 85%.

31. The absorbent article according to claim 1 having a Utilization Rate greater than about 90%.

30. The absorbent article according to claim 1 having a Utilization Rate greater than about 95%.
